SEQUENCE LISTING (7 sequences)

SEQ ID NO: 1 - DNA, 32 bp, Artificial Sequence
  Synthetic Oligonucleotide
aaaaaaaaaatatgacagaactagacactctt

SEQ ID NO: 2 - DNA, 35 bp, Artificial Sequence
  Synthetic Oligonucleotide
aaaaaaaaaagacgctaatagttaagacgcttuuu

SEQ ID NO: 3 - DNA, 65 bp, Artificial Sequence
  Synthetic Oligonucleotide
ctattagcgtccagtgaatgcgagtccgtctaagagagtagtacagcagccgtcaagagtgtcta

SEQ ID NO: 4 - DNA, 25 bp, Artificial Sequence
  Synthetic Oligonucleotide
gttctgtcatatttaagcgtcttaa

SEQ ID NO: 5 - DNA, 20 bp, Artificial Sequence
  Synthetic Oligonucleotide
cagtgaatgcgagtccgtct

SEQ ID NO: 6 - DNA, 26 bp, Artificial Sequence
  Synthetic Oligonucleotide
aaaaaacagtgaatgcgagtccgtct

SEQ ID NO: 7 - DNA, 29 bp, Artificial Sequence
  Synthetic Oligonucleotide
aaaaaaaaacagtgaatgcgagtccgtct

(19) United States
(12) Patent Application Publication
(10) Pub. No.: US 2013/0323729 A1
(43) Pub. Date: Dec. 5, 2013

(54) Proximity Ligation Technology for Western Blot Applications

(71) Applicants: Ulf Landegren, Uppsala (SE); Åsa Hagner-McWhirter, Uppsala (SE); Daniel Ivansson, Uppsala (SE)

(72) Inventors: Ulf Landegren, Uppsala (SE); Åsa Hagner-McWhirter, Uppsala (SE); Daniel Ivansson, Uppsala (SE)

(73) Assignee: OLINK AB, Uppsala (SE)

(21) Appl. No.: 13/882,228

(22) PCT Filed: Oct. 27, 2011
(86) PCT No.: PCT/SE11/51279

§ 371 Date: Aug. 9, 2013

Related U.S. Application Data
(60) Provisional application No. 61/407,944, filed on Oct. 29, 2010.
(60) Provisional application No. 61/411,955, filed on Nov. 10, 2010.

(51) Int. Cl. G01N 33/543

(52) U.S. Cl. CPC G01N 33/5436
USPC 435/6.11

(57) ABSTRACT

The invention provides a method for detecting a biomolecular feature (a protein, protein complex, or modified protein such as a phosphorylated protein) by a modified Western blot type of assay, which method either electrophoretic gel separation followed by transfer, or direct spotting of a sample containing the biomolecular feature onto a membrane; providing a proximity probe pair, each probe comprising a binding moiety with affinity for a different binding site on the bio molecular feature and a reactive oligonucleotide, coupled thereto; binding the proximity probes to their respective binding sites on the biomolecular feature through the binding moiety, adding a splint oligonucleotide and a backbone oligonucleotide which are complementary to the reactive oligonucleotide pair, and allowing hybridization among them; ligating the hybridized DNA oligonucleotides to create a circularized DNA molecule when both probes bind sufficiently close to each other on the bio molecular feature, amplifying the circularized DNA by isothermal amplification; and detecting the presence and quantity of the bio molecular feature using a detection oligonucleotide complementary to the amplification product. Also provided are methods for multiplexed detection of more than one bio molecular feature, as well as kits for performing the assays.

### FIELD OF THE INVENTION

The present invention relates to improved methods for Western blot type of analysis. More specifically, the invention relates to a Western blot method which incorporates the advantages of the proximity ligation technology. Also provided is a multiplexed Western blot method using the proximity ligation technology.

### BACKGROUND OF THE INVENTION

### Western Blotting

Western blotting (or, protein immunoblotting) is an analytical technique used to detect specific proteins in a given sample of tissue homogenate, cell lysate or other protein containing samples. It uses gel electrophoresis to separate native or denatured proteins by the length of the polypeptide (denaturing conditions) or by the 3-D structure of the protein (native/non-denaturing conditions). The proteins are then transferred to a membrane (typically nitrocellulose or PVDF), where they are probed (detected) using antibodies specific to the target protein (see http://en.wikipedia.org/wiki/Western_blot). Another option similar to Western blotting is to perform a Dot Blot or reverse phase protein microarray assay (RPMA), were the protein samples are directly spotted onto a membrane without an electrophoresis separation step. For certain applications, the proteins are probed in the gel without a transfer step. However, further description of Western blot methodology is based on protein samples transferred to a membrane. During the detection process the membrane is probed for the protein of interest by the use of an antibody specific for the protein of interest. Due to possibilities of increased signal amplification and to avoid negative effects on target specific affinity related to primary antibody conjugation, this traditionally takes place in a two-step process (using a primary target specific antibody and a secondary labeled antibody specific for the primary antibody), although there are now one-step detection methods available for certain applications. The one-step method allows the process to occur faster and with a lower amount of consumables, but sensitivity may be compromised. This requires a probe antibody which both recognizes the protein of interest and contains a detectable label, probes which are often available for known protein tags. The primary probe is incubated with the membrane in a manner similar to that for the primary antibody in a two-step process, and is then ready for direct detection after a series of wash steps.

There are a number of detection methods available for Western blotting, here illustrated for secondary detection formats.

The colorimetric detection method depends on incubation of the Western blot with a substrate that reacts with the reporter enzyme (such as peroxidase or alkaline phosphatase) that is conjugated to the secondary antibody. This converts the soluble dye into an insoluble form of a different color that precipitates next to the enzyme and thereby stains the membrane. Development of the blot is then stopped by washing away the soluble dye. Protein levels are evaluated through densitometry (how intense the stain is) or spectrophotometry.

Chemiluminescent detection methods depend on incubation of the membrane/blot with a substrate that will luminesce when exposed to the reporter enzyme on the secondary antibody. The light signal is then detected by photographic film, and more recently by CCD cameras which capture a digital image of the membrane/blot. Image analysis software is used to quantify the signals and for calculation of molecular weights (MW) if a MW standard was run on the gel together with the samples.

Radioactive labeled secondary antibodies are also used and the signals are detected by using X-ray film of phospho imaging screens. The importance of radioactive detections methods is declining, because it is very expensive, health and safety risks are high and ECL provides a useful alternative (see http://en.wikipedia.org/wiki/Western_blot).

Fluorescently labeled secondary antibodies are detected by fluorophore specific excitation wavelength and emission filter settings using a laser or CCD based imager. Fluorescence is considered to be among the most sensitive detection methods for blotting analysis and has the advantage of multiplexing possibilities, enabling detection of targets of the same molecular weight (overlaid signals distinguished by using separate detection channels).

### Proximity Ligation Assay

In-situ PLA (proximity ligation assay) technology was developed by Ulf Landegren et al. (In situ detection of phosphorylated PDGF receptor β using a generalized proximity ligation method; Jarvius, M., et al.; Mol Cell Proteomics. 2007 September; 6(9):1500-9. Epub 2007 Jun. 12) and commercialized by Olink Biosciences AB (www.olink.se). In-situ PLA offers extreme signal amplification and the possibility to count individual binding events (when microscope read-out is used). Via the optional use of dual recognition events at the primary level, the specificity is highly increased. This detection principle has been applied to interrogation of fixed tissue/cells (immunohistochemistry-like applications) and to a lesser extent protein arrays.

In the standard design, two affinity-binders (antibodies, affibodies, aptamers etc.) are conjugated to sequence-designed oligonucleotides and used to probe a sample (FIG. 1). If and only if the two reagents bind in proximity of each other a paired set of specialized and sequence matched oligonucleotides (i.e. backbone- and splint oligo) can hybridize to the binder-conjugated oligos and be converted to a circular molecule by ligation reactions. Next, rolling circle amplification (RCA) is used to elongate one of the binder-conjugated oligos. As a result, each correctly bound pair of affinity reagents are converted into localized DNA-spheres (˜1 μm in diameter, also referred to as rolling circle products or RCPs) containing up to a thousand copies of the circular DNA molecule (engineered to contain binding sites for oligonucleotide reporter probes). The detection is accomplished through hybridization of detection oligos. More advanced designs requiring three or more oligo-conjugated affinity-reagents binding in proximity for circularization/RCA have also been reported (Protein Diagnostics by Proximity Ligation: Combining Multiple Recognition and DNA Amplification for Improved Protein Analyses, Leuchowius, K-L., et al., Molecular Diagnostics (Second Edition), 2010, Pages 299-306).

There are several possible implementations of the standard design, for example: (1) A single primary antibody in combination with two oligo-conjugated secondary antibodies. The secondary antibodies specifically recognize two distinct epitopes of the primary antibody (species specific and/or conjugated haptens such as biotin). (2) Two primary antibodies in combination with two oligo-conjugated secondary antibodies. Primary antibodies need to be of different species origin or conjugated to different haptens. (3) Two oligo-conjugated primary antibodies.

### SUMMARY OF THE INVENTION

In a first aspect of the invention, it is provided a method for detecting a bio molecular feature (i.e., a protein, a protein complex, or a modified protein such as a phosphorylated protein) by a modified Western blot type of assay. The method comprises (a) detection of a bio molecular feature in a sample on a porous membrane format such as electrophoretic gel separation followed by transfer or direct spotting of a sample containing the bio molecular feature to a membrane; (b) providing a proximity probe pair, each probe comprising a binding moiety with affinity for a different binding site on the bio molecular feature and a reactive oligonucleotide, coupled thereto; (c) binding the proximity probes to their respective binding sites on the bio molecular feature through the binding moiety; (d) adding a splint oligonucleotide and a backbone oligonucleotide which are complementary to the reactive oligonucleotide pair, and allow them to hybridize, thereby bringing the ends of the backbone and splint oligonucleotides in direct contact; (e) ligating, using a DNA ligase, the hybridized DNA to create a circularized DNA molecule wherein the circularized DNA molecule is formed from the backbone and splint oligonucleotides only when the probes in the proximity probe pair bind sufficiently close to each other on the bio molecular feature; (f) elongating one of the reactive oligonucleotides by isothermal amplification using the circularized DNA molecule as template, thereby creating a localized amplification product; (g) detecting the presence and quantity of the bio molecular feature using a detection oligonucleotide complementary to the amplification product.

In certain embodiments, the binding moieties are antibodies and the antibodies each bind to the bio molecular feature via the aid of one or two primary antibodies having direct binding specificity for the bio molecular features, and the binding moieties are directed against the Fc portion or conjugated haptens of the further antibody/antibodies.

In other embodiments, the binding moieties of the proximity probes have direct specificity for epitopes of the bio molecular feature and are selected from a protein, such as a monoclonal or polyclonal antibody, lectin, soluble cell surface receptor, combinatorially derived protein from phage display or ribosome display, peptide, carbohydrate, nucleic acid, such as an aptamer, or combinations thereof.

In still other embodiments, the isothermal amplification is rolling circle amplification. Preferably, the rolling circle amplification is performed using Phi29 DNA polymerase.

In some embodiments, the sample is a homogenized tissue or cell lysate, a body fluid, or cell culture supernatant.

In one embodiment, the binding between the proximity probes and the bio molecular features as well as the detection are performed directly in the electrophoretic gel. In other embodiments, the sample is either separated in an electrophoretic gel and transferred to a suitable blotting membrane, such as a nitrocellulose or PVDF membrane, or directly spotted onto such a membrane without separation before binding and detection with the proximity probes.

In certain embodiments, the detection oligonucleotides are fluorescently labeled and the detection is performed by fluorescence read-out. In certain other embodiments, the detection oligonucleotides are labeled with an HRP enzyme and the detection is performed by ECL read-out.

In a second aspect of the invention, it is provided a method for multiplexed detection of two or more target bio molecular features (i.e., a protein, a protein complex, or a modified protein such as a phosphorylated protein) by a modified Western blot type of assay. The method comprises (a) detection of a bio molecular feature in a sample on a porous membrane format such as electrophoretic gel separation followed by transfer or direct spotting of a sample containing the bio molecular feature to a membrane; (b) for each of the target bio molecular features, providing a proximity probe pair, each probe comprising a binding moiety with affinity for a different binding site on the bio molecular feature and a reactive oligonucleotide, coupled thereto; (c) binding the proximity probes to their respective binding sites on each of the bio molecular features through the binding moiety; (d) adding oligonucleotides which are complementary to the reactive oligonucleotide pair, and allow them to hybridize, thereby bringing the ends of the oligonucleotides in direct contact; wherein when both proximity probes bind to the same bio molecular feature in proximity, a circularized DNA molecule is formed and an amplification product (i.e., rolling circle product) can be generated in subsequent steps, further wherein the amplification product from each proximity probe pair carries at least one unique detection sequence site, which is unique from a detection sequence site in an amplification product for a different target bio molecular feature; (e) ligating, using a DNA ligase, the hybridized DNA to create a circularized DNA molecule wherein the circularized DNA molecule is formed only when the probes in the proximity probe pair bind sufficiently close to each other on the bio molecular feature; (f) elongating one of the reactive oligonucleotides by isothermal amplification using the circularized DNA molecule as template, thereby creating a localized amplification product; (g) providing, for each target bio molecular feature, a detection oligonucleotide mixture based on a sequence which is complimentary to the unique detection sequence site on the amplification product; wherein the detection oligonucleotide mixture for the target bio molecular feature contains a defined ratio of species with identical sequence but labeled with different fluorescent dyes such that during detection, a unique signal ratio is associated with the amplification product for the bio molecular feature; (h) pooling the detection oligonucleotide mixtures for all the target bio molecular features and hybridizing the detection oligonucleotide mixtures with the amplification products; (i) detecting the signal from each label for each of the two or more different labels; (j) generating a signal gain calibration for the different labels using a predefined standard sample present on a defined and spatially separated region of the reaction volume; (k) calculating the ratio of labels for each location using the signal gain calibration and detecting the target bio molecular features of interest based on the detection of the unique signal ratios.

In certain embodiments, the method further comprising: for each of the target bio molecular features, providing one splint oligonucleotide and one backbone oligonucleotide which are complementary to the reactive oligonucleotides, thus a circularized DNA molecule is formed through hybridization and ligation of these oligonucleotides, provided both proximity probes bind to the same bio molecular feature in proximity.

In certain embodiments, the binding moieties are antibodies and the antibodies each bind to the bio molecular feature via the aid of one or two further antibody/antibodies having direct binding specificity for the bio molecular feature, and wherein the binding moieties are directed against the Fc portion or conjugated haptens of the further antibody/antibodies.

In other embodiments, the isothermal amplification is rolling circle amplification. Preferably, the isothermal amplification is performed using Phi29 DNA polymerase.

In still other embodiments, the binding moieties of the proximity probes have direct specificity for the bio molecular feature and are selected from a protein, such as a monoclonal or polyclonal antibody, lectin, soluble cell surface receptor, combinatorially derived protein from phage display or ribosome display, peptide, carbohydrate, nucleic acid, such as an aptamer, or combinations thereof.

In some embodiments, the sample is a homogenized tissue or cell lysate, a body fluid, or cell culture supernatant.

In certain embodiments, the binding between the proximity probes and the bio molecular feature as well as the detection are performed directly in an electrophoretic gel.

In other embodiments, the detecting step is performed by taking multiple scans/images at different excitation wavelength/emission filter combinations. In still other embodiments, the detecting step is performed by multi-spectral imaging (i.e. by recoding complete emission spectra in each pixel).

In another aspect of the invention, it is provided a kit for proximity ligation assay based Western blot type of analysis. The kit comprises a proximity probe pair, each probe comprising a binding moiety with affinity for a different binding site on a bio molecular feature and an oligonucleotide acting as a reactive oligonucleotide, coupled thereto; a splint oligonucleotide and a backbone oligonucleotide which are complementary to the reactive oligonucleotide pair; a detection oligonucleotide; one or more optimized buffers, enzymes and protocols. In certain embodiments, the detection oligonucleotide is labeled. In other embodiments, the binding moieties are antibodies and the antibodies each bind to the bio molecular feature via the aid of one or two further antibody/antibodies having direct binding specificity for the bio molecular feature, and wherein the binding moieties are directed against the Fc portion or conjugated haptens of the further antibody/antibodies. In still other embodiments, the kit further comprises a DNA ligase and an enzyme for isothermal amplification, such as rolling circle amplification.

In a further aspect of the invention, it is provided a kit for multiplexed Western blot analysis of two or more target bio molecular features. The kit comprises, for each of the target bio molecular features: a proximity probe pair, each probe comprising a binding moiety with affinity for a different binding site on the bio molecular feature and an oligonucleotide acting as a reactive oligonucleotide, coupled thereto; a splint oligonucleotide and a backbone oligonucleotide which are complementary to the reactive oligonucleotide pair, thus a circularized DNA molecule can be formed and an amplification product can be generated during the reaction process provided both proximity probes bind to the same bio molecular feature in proximity; a detection oligonucleotide mixture based on a sequence which is complimentary to a unique detection sequence site on the amplification product; wherein the detection oligonucleotide mixture for each target bio molecular feature contains a defined ratio of species with identical sequence but labeled with different fluorescent dyes such that during detection, a unique signal ratio is associated with the amplification product for each substrate; the kit further includes a standard sample for calibration of fluorescent dye signal gains; one or more optimized buffers; and protocols. In one embodiment, the kit further comprises a DNA ligase and an enzyme for isothermal amplification, such as rolling circle amplification. In another embodiment, the binding moieties are antibodies and the antibodies each bind to the bio molecular feature via the aid of one or two further antibody/antibodies having direct binding specificity for the substrate, and wherein the binding moieties are directed against the Fc portion or conjugated haptens of the further antibody/antibodies. In another embodiment, the binding moieties have direct specificity for the bio molecular feature and are selected from a protein, such as a monoclonal or polyclonal antibody, lectin, soluble cell surface receptor, combinatorially derived protein from phage display or ribosome display, peptide, carbohydrate, nucleic acid, such as an aptamer, or combinations thereof.

### BRIEF DESCRIPTION OF THE DRAWINGS

FIG. 1 shows a schematic overview of standard in-situ PLA implementations of prior art.

FIGS. 2A, 2B and 2C show proof of principle, showing increased signal intensity for PLA, compared to ECL™ Plex, using Dot blot and fluorescence detection.

FIGS. 3A and 3B show the increased signal intensity for tubulin in cell lysates using PLA Western blot compared to traditional Western blotting with chemiluminescence detection.

FIGS. 4A and 4B show the increased specificity for dual recognition PLA Western compared to either traditional Western or single recognition PLA Western of both tubulin and Villin protein targets with chemiluminescence detection, in a cell lysate sample.

FIGS. 5A and 5B show direct single signal detection of protein modification with PLA Western compared to traditional Western blotting using chemiluminescence detection.

FIG. 6 show an overview of the multiplexing design using ratio-mixed fluorescent detection probes according to one embodiment of the invention.

FIG. 7 shows an example of the multiplexing design according to one embodiment of the invention.

FIG. 8 shows resolution of ratio-mixed fluorescent dyes using a fluorescent scanner.

### DETAILED DESCRIPTION OF THE INVENTION

### In Situ PLA Western Blot

One embodiment of the invention provides an improved method for Western blot applications. In particular, it is provided a procedure for using PLA in Western blot applications. For the first time the Applicants have shown that PLA can be used in a Western blot application.

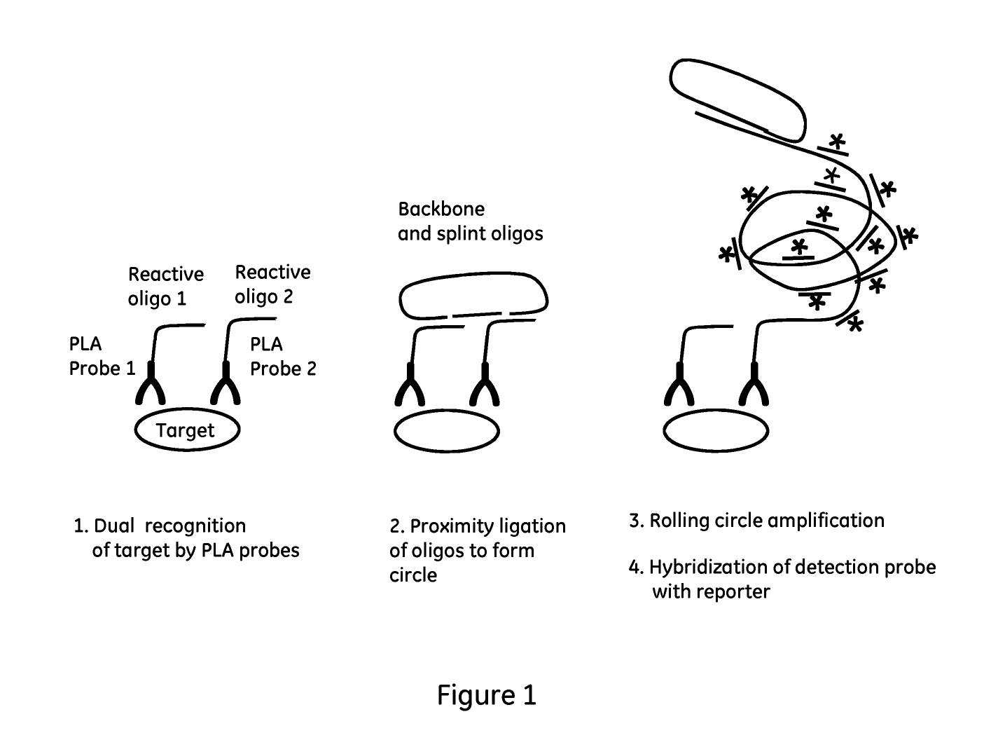

The detection scheme is based on in-situ PLA. Here, two PLA probes (oligo-conjugated antibodies or other affinity reagents) are used to probe the Western blot or Dot blot membrane (or the electrophoretic gel, in case of In-gel Western applications) for binding to two distinct protein epitopes (primary detection format) or two distinct primary antibody epitopes (for detection of a protein, protein modification or protein-protein interaction). If the two PLA-probes bind in proximity of each other they can prime the creation of a circularized DNA molecule. This is achieved by addition of a backbone oligo and a splint oligo (that are complementary to the PLA-probe oligos) and a ligase. The sequences are designed such that in the next step the circularized DNA molecule is bound only to one of the PLA probes. A DNA polymerase capable of strand displacement amplification (e.g., Phi29 DNA polymerase) and nucleotides are added and the PLA probe oligo is elongated via rolling circle amplification. In the final step the localized RCPs (rolling circle products) are detected via detection oligos either conjugated to horseradish peroxidase HRP for ECL read-out or to a fluorescent dye for fluorescent read-out (FIG. 1).

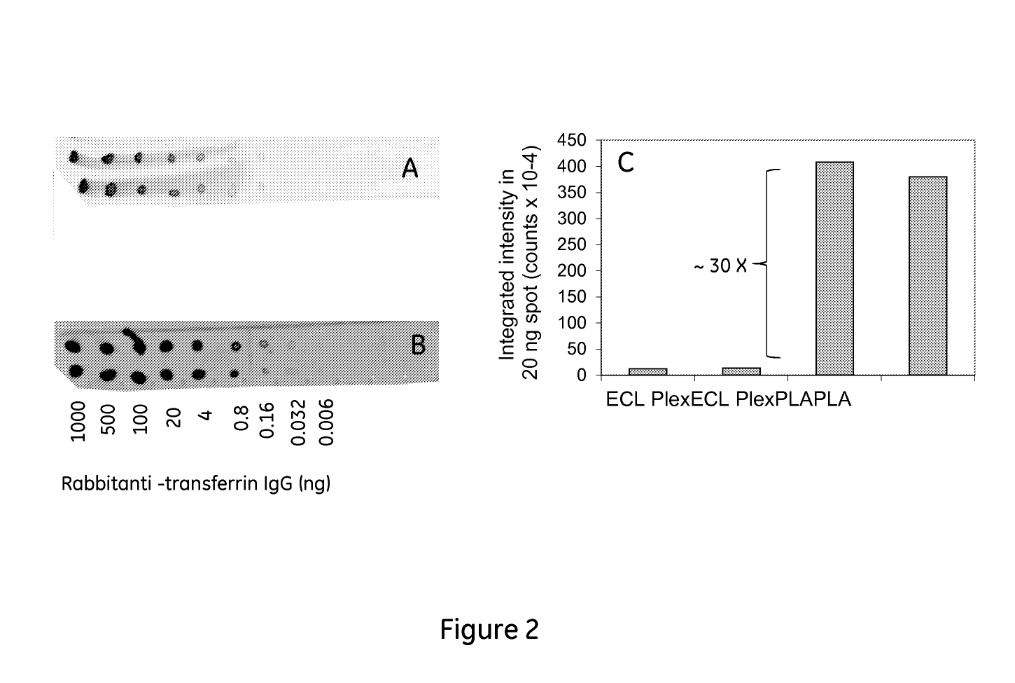

As proof of principle, the sensitivity of PLA was investigated using dot blot. For comparison, parallel experiments with ECL™ Plex detection have been done. FIG. 2 shows signal intensity of the PLA method, compared to ECL™ Plex, using CY™5 detection, of a dilution series of rabbit anti-transferrin antibody. The results show approximately 30 fold increase in signal intensity for PLA detection compared to ECL™ Plex detection.

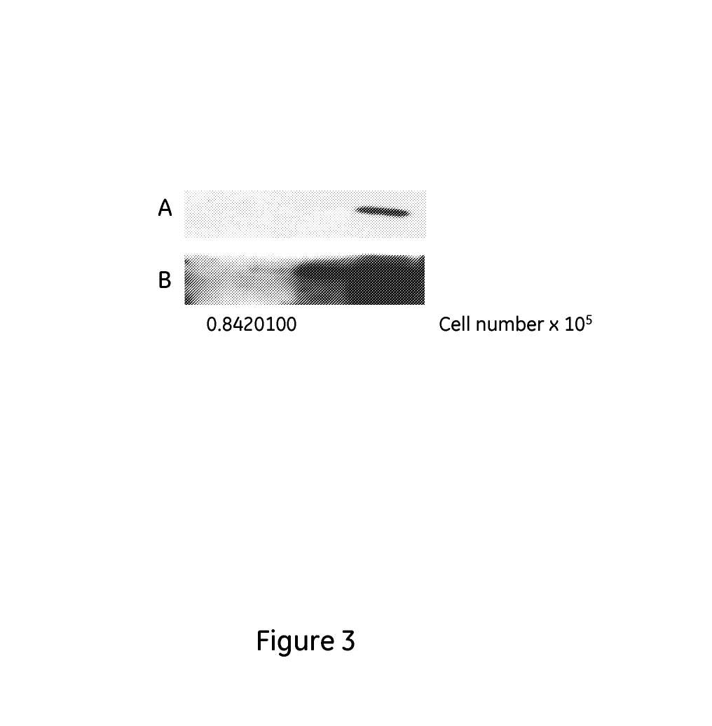

The sensitivity of PLA was shown by increased signal intensity in Western blot assays. A cell lysate dilution series was analyzed by Western blot, using chemiluminescence detection. Endogenous tubulin was detected by primary mouse anti-tubulin and secondary HRP conjugated antibody and ECL or PLA HRP/ECL detection. A 25-fold increase in signal intensity was observed for PLA (FIG. 3).

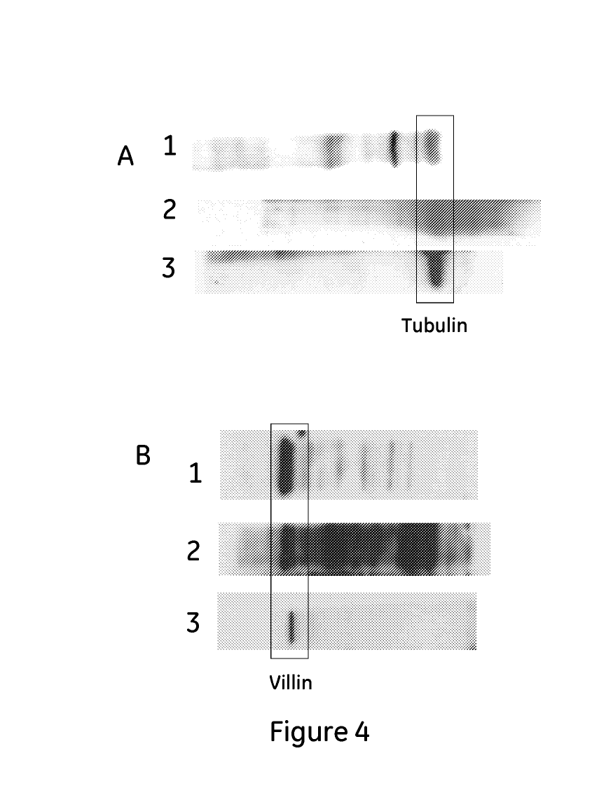

The specificity can be improved with removal of unspecific detection caused by poor quality primary antibodies. Using poor quality antibodies alone (single detection), give unspecific detection of protein bands whereas combining two poor quality primary antibodies using dual recognition PLA Western gives increased specificity in detection. Increased specificity was achieved for tubulin detection using dual recognition PLA Western compared to traditional ECL Western detection (FIG. 4A). Specificity of Villin detection was highly improved using dual recognition PLA Western, compared to single recognition PLA Western using a liver tissue sample (FIG. 4B).

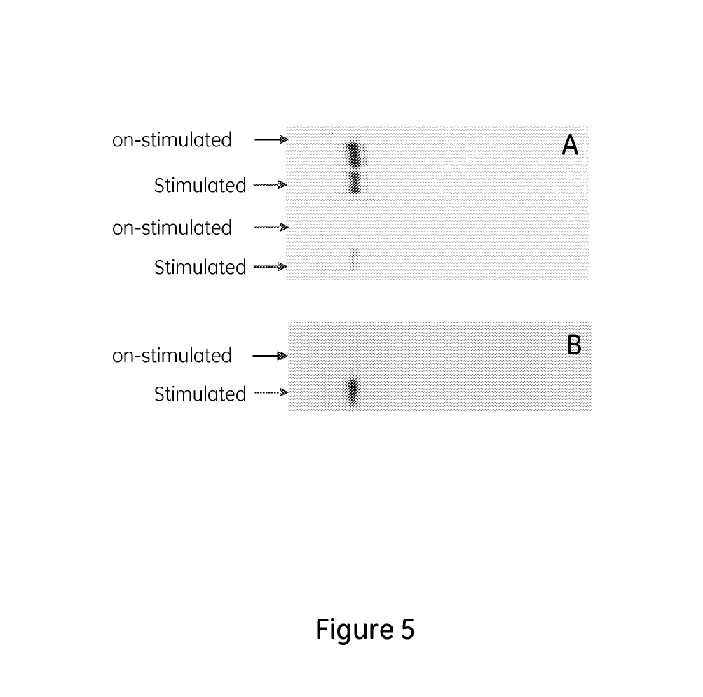

To assign a phosphorylation to a particular protein in current Western blot assays, signals from both a protein specific antibody and a phospho-epitope specific antibody need to be detected as two overlapping signals detected in separate channels (fluorescence), in separate lanes or blots (chemiluminescence). Using PLA Western, improved detection possibilities can be achieved by combining the two antibodies to generate a single signal if they bind in proximity (on the same protein). Detection of phosphorylated PDGFRβ with a single signal using PLA Western is achieved compared to traditional Western, where detection of two signals is needed (total protein and phosphorylated protein, see FIG. 5).

In certain embodiments, the invention also provides a kit for Western blot analysis, which kit comprises a proximity probe pair each probe comprising a binding moiety with affinity for a different binding site on a bio molecular feature (protein, protein complex or a specifically modified protein such as a phosphorylated protein) and an oligonucleotide acting as a reactive functionality (reactive oligonucleotide), coupled thereto; a splint oligonucleotide and a backbone oligonucleotide which are complementary to the reactive oligonucleotide; a detection oligonucleotide, one or more optimized buffers, and protocols. Optionally, the detection oligonucleotide is labeled. Optionally, the kit further comprises a DNA ligase and an enzyme for isothermal amplification, such as rolling circle amplification. As an example, Phi29 DNA polymerase is included as the enzyme for isothermal amplification.

In certain embodiments, the binding moieties are antibodies and the antibodies each bind to the bio molecular feature via the aid of one or two further antibody/antibodies having direct binding specificity for the bio molecular feature, and wherein the binding moieties are directed against the Fc portion and/or conjugated haptens of the further antibody/antibodies.

In other embodiments, the binding moieties have direct specificity for epitopes of the bio molecular feature and are selected from a protein, such as a monoclonal or polyclonal antibody, lectin, soluble cell surface receptor, combinatorially derived protein from phage display or ribosome display, peptide, carbohydrate, nucleic acid, such as an aptamer, or combinations thereof.

The use of in-situ PLA (proximity ligation assay) technology for Western blotting applications has been shown here to greatly increase the sensitivity and at the same time improve the specificity by the optional requirement of two proximal binding events at the primary level. For Western blotting, the benefit of increased sensitivity with the possibility to detect low abundant proteins in small amounts of samples cannot be overstated. For example, analysis of scarce samples (isolated stem cells/primary cells, xenograft aspirates and biopsies) not amendable to Western blot analysis previously is now within reach. Further, the increased sensitivity potentially enables improved In-gel Western blot analysis (detection performed in a near-surface volume of electrophoretic gels), thereby speeding up analysis and removing potential variation introduced during the blotting step.

Via the use of dual recognition events at the primary level, the specificity is highly increased. Due to this high specificity using dual recognition detection it would be possible in some applications to omit the electrophoresis size separation step before protein analysis and directly spot the sample on a membrane (Dot blot). By using dual recognition, only the target bio molecular feature will be detected and unspecific signals will not be generated. Traditionally, a size separation and molecular weight confirmation is needed to avoid the risk of unspecific detection at the migration position of the target protein ensuring analysis of true signals. The high specificity using dual recognition can also be exploited to extend the range of usable antibodies (and thereby targets) or to design unique assays detecting protein specific posttranslational modifications (such as phosphorylations, glycosylations or acetylations) or stable protein interactions (using native gel electrophoresis conditions) with high sensitivity and specificity.

### Multiplex of In Situ PLA Western Blot

Based on the successful application of in-situ PLA in Western blot, another aspect of the invention provides a method for controlling/building localized fluorescent bar-codes based on combinations of target specific RCPs and fluorophore labeled detection oligonucleotides. This enables a procedure for increasing the obtainable multiplexing-level in Western blots without the need of a high number of fluorophores or the use of stripping methodologies or other repeated probing strategies. In addition the high sensitivity and unique detection specificity offered by PLA Western blot can thus be exploited for multiple targets at once, thereby further extending the amount and type of information that can be extracted from scarce samples (isolated stem cells/primary cells, xenograft aspirates and biopsies).

The following features of in-situ PLA are essential to this aspect of the invention:

(1) Fluorophores are administered via detection oligonucleotides. In contrast to antibody labeling this enables precise control over the number of fluorophore molecules per reagent (i.e. one).
(2) A large number of target sequence repeats (˜1000) are present (in target specific RCPs) locally for each specifically detected antigen. Hence, variation in actual fluorophore ratios for individual RCPs, due to statistical effects during hybridization, can be kept low.

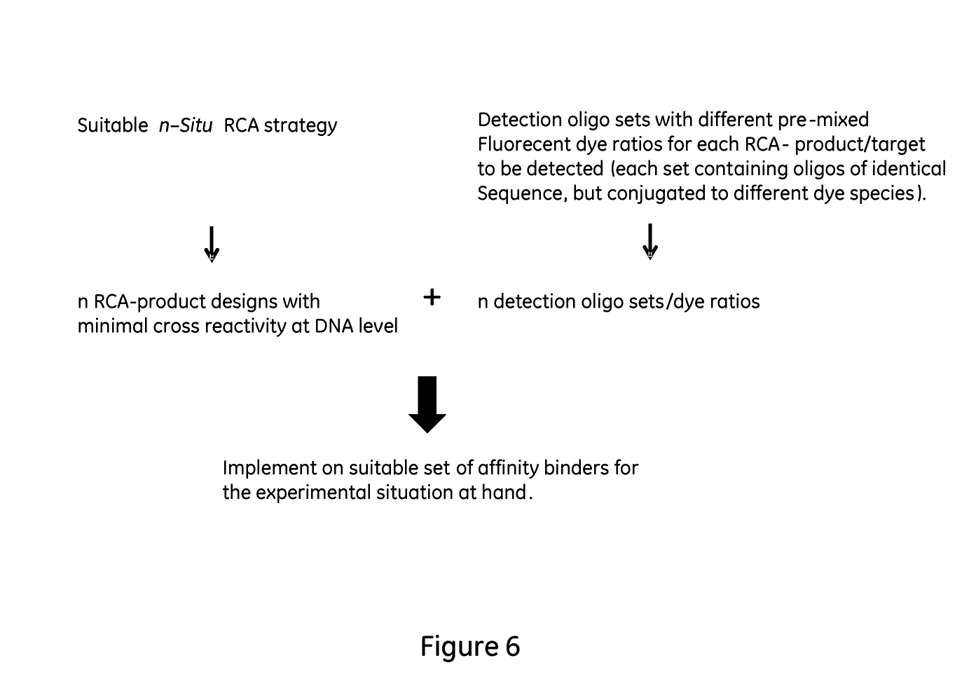

Thus, one embodiment of the invention provides a multiplexed in situ PLA-based Western blot method, using unique bar-codes based on defined ratios of two (or more) fluorophores in target specific RCPs (FIG. 6). In this method, oligonucleotide sets (two oligos for creation of PLA probes via conjugation to affinity binders, plus one backbone and one splint oligo) for the number of targets to be detected is designed for minimal cross-reactivity during proximity ligation and RCA. Hence, using suitable affinity reagents, target specific RCPs can be generated during reactions on a Western blot membrane. Further, each target specific RCP is designed to contain amplified copies of a target specific sequence region which can promote hybridization to a unique detection oligonucleotide sequence. For each RCP a sequence complementary to the unique detection sequence is produced. Each such detection oligonucleotide sequence is conjugated to one or more different fluorescent dyes (in case of multiple dyes, different aliquots of unlabeled oligonucleotides are conjugated to single dyes). For each target bio molecular feature/RCP to be detected, defined and unique ratios of the differently labeled detection oligonucleotides (all having identical sequence) are pre-mixed (one mixture for each target bio molecular feature/unique RCP). The complete set of detection oligonucleotide mixtures are added to the reaction containing the membrane, and the dye ratios are transferred to localized target specific RCPs via sequence specific hybridization. A fluorescent imager or scanner is used to generate multiple images (one with optimal settings for each dye included) of the membrane. De-coding is dependent on proper calibration of signal gains for the different dyes included. For the purpose of image acquisition equipment calibration, a well-defined standard sample (probed using one of the RCP designs used for unknown samples and an equimolar mixture of all the dyes included in the experiment) is present at a known and spatially separated location on the membrane. Following calibration, measured dye ratios for different locations on the membrane can be de-coded. Quantitative analysis is then performed using optimal images for each target.

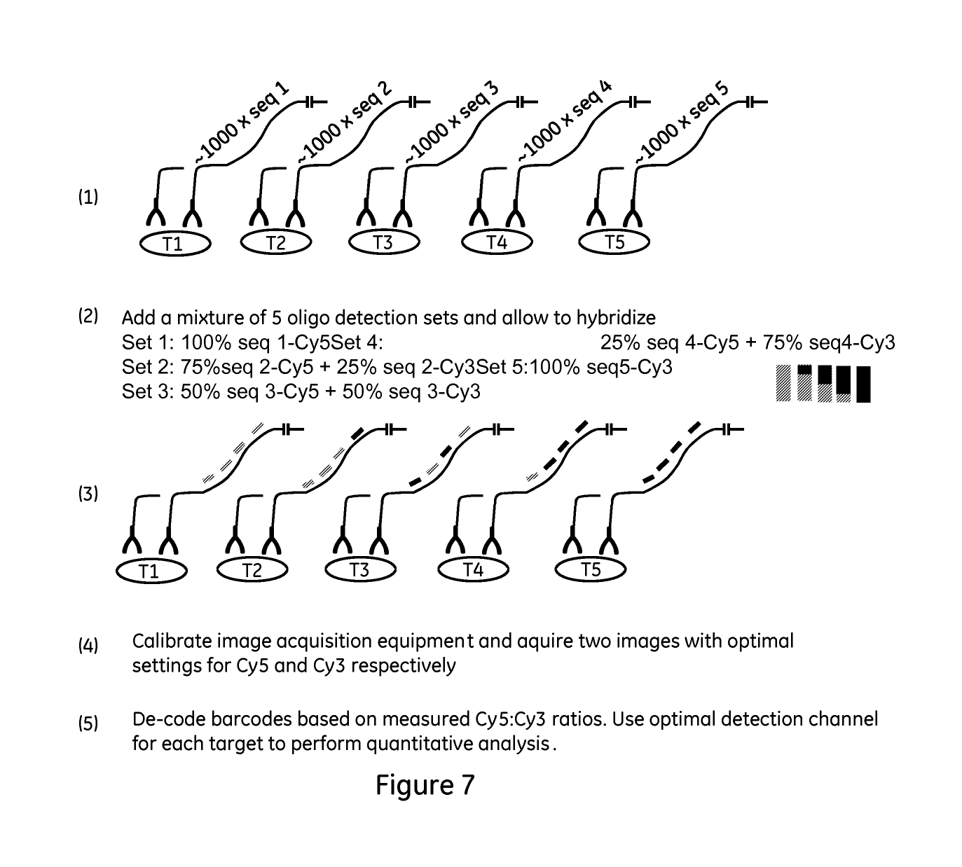

FIG. 7 provides an example of a 5-plex PLA Western blot using only CY™3 and CY™5, with localized “bar-codes”:

(1) Each target bio molecular feature to be analyses is assigned a bar-code based on a defined ratio of CY™5 to CY™3 (only CY™5, 3:1, 1:1, 1:3 and only CY™3). Five PLA reagent sets (each containing two PLA probes and corresponding backbone-, splint- and detection oligonucleotides) are designed according to the scheme outlined in the general description. Each set recognizes a unique target bio molecular feature on a Western membrane and generates target specific RCPs, with minimal interference from other sets, following proximity ligation and RCA.
(2) Aliquot(s) of each detection oligonucleotide are labeled with CY™5, CY™3 or both and then mixed in the predetermined CY™5/CY™3 ratio. Thus the group of five detection oligonucleotide sequences are present in the following mixtures: (1) 100% CY™5; (2) 3:1 of CY™5: CY™3; (3) 1:1 of CY™5: CY™3; (4) 1:3 of CY™5: CY™3; and (5) 100% CY™3; respectively.
(3) Detection oligonucleotides for the five target bio molecular features are mixed together, added to the membrane and thereby transferred to corresponding local RCPs via sequence-specific hybridization.
(4) A suitable image acquisition equipment is used to generate images with optimal settings for CY™5 and CY™3, respectively. Calibration of CY™5 to CY™3 signal gain is achieved via the procedure outlined in the general description (using a spatially separated standard sample).
(5) The “barcodes” are decoded based on the signal gain calibration and measured CY™5:CY™3 ratios. Furthermore, optimal detection channels are used for each target to perform quantitative analysis (channel/image with highest signal to noise chosen for each target/barcode).

Thus, as shown in FIG. 7, following proximity ligation and RCA each unique target bio molecular feature (e.g., protein, protein modification or protein complex) can be detected using an oligonucleotide the sequence of which corresponds to a complimentary sequence of the corresponding RCP. The oligonucleotides are labeled with either CY™5 or CY™3, but the combined oligonucleotides pool for each target has a predetermined ratio of CY™5 and CY™3. The oligonucleotide pool of oligonucleotides for each target has a ratio of CY™5 and CY™3 distinguishable from the ratios of other oligonucleotides pools. Thus, multiplexing is achieved. Although FIG. 7 presents an example with two dyes, it could be advantageous in certain circumstances to use more than two dyes. The principle is similar nonetheless.

An optimized set of bar-code oligonucleotide sets as outlined above can be transferred to any desired set of antibodies (or other affinity reagents) and used in different assay set-ups. To reach really high multiplexing, oligonucleotide conjugation needs to be performed at the primary binder level due to the limited number of different sources (species) of antibodies available. However, the principle can also be applied to secondary detection formats. For example to design a 5-plex secondary kit using only two fluorophores.

In certain embodiments, the invention also provides a kit for multiplexed Western blot analysis of two or more target bio molecular features, comprising: for each of the target substrates, a proximity probe pair, each probe comprising a binding moiety with affinity for a different binding site on the bio molecular feature and an oligonucleotide acting as a reactive functionality (reactive oligonucleotide), coupled thereto; a splint oligonucleotide and backbone oligonucleotide which are complementary to the reactive oligonucleotides, thus a circularized DNA molecule can be formed and an amplification product can be generated during the reaction process, provided both proximity probes bind to the same bio molecular feature in proximity; a detection oligonucleotide mixture based on a sequence which is complimentary to a unique detection sequence site on the amplification product; wherein the detection oligonucleotide mixture for each target bio molecular feature contains a defined ratio of species with identical sequence but labeled with different fluorescent dyes such that during detection, a unique signal ratio is associated with the amplification product for each bio molecular feature, the kit further includes a standard sample for calibration of fluorescent dye signal gains, one or more optimized buffers, and protocols. Optionally, the kit further comprises a DNA ligase and an enzyme for isothermal amplification, such as rolling circle amplification. As an example, Phi29 DNA polymerase is included as the enzyme for isothermal amplification.

In certain embodiments, the binding moieties are antibodies and the antibodies each bind to the bio molecular feature via the aid of one or two further antibody/antibodies having direct binding specificity for the bio molecular feature, and wherein the binding moieties are directed against the Fc portion and/or conjugated haptens of the further antibody/antibodies.

In other embodiments, the binding moieties have direct specificity for the bio molecular feature and are selected from a protein, such as a monoclonal or polyclonal antibody, lectin, soluble cell surface receptor, combinatorially derived protein from phage display or ribosome display, peptide, carbohydrate, nucleic acid, such as an aptamer, or combinations thereof.

### Examples

The invention will now be more fully described in association with some examples which are not to be construed as limiting for the invention.

### Experimental

### Electrophoresis and Transfer:

Standard electrophoresis (miniVE Vertical Electrophoresis System, GE Healthcare) and wet transfer (TE 22 Mini Tank Transfer Unit, GE Healthcare) protocols were used according to manufactures instructions.

### Protein Samples

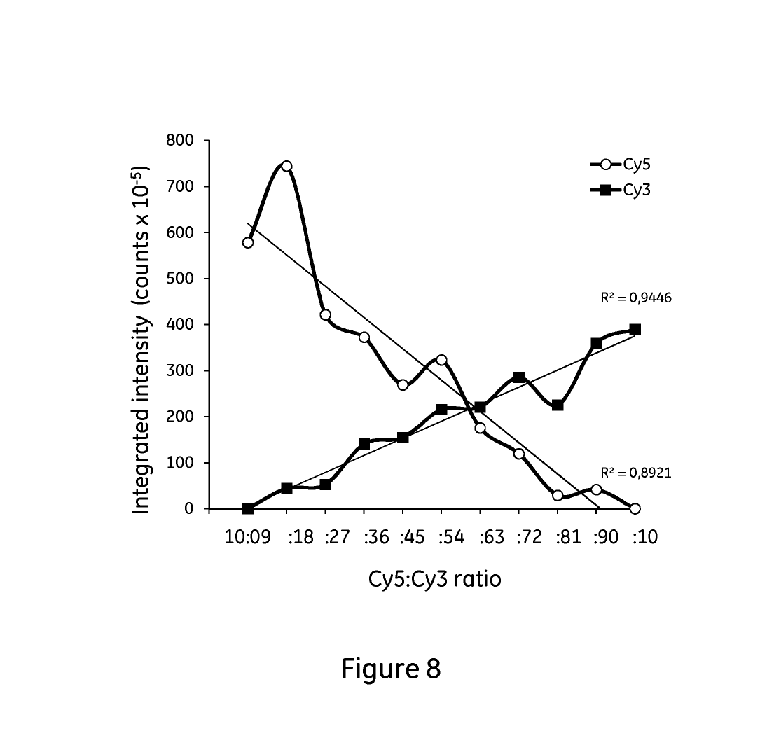

Human fibroblast cell lysate (cell line BJh-TERT).
Rabbit anti-transferrin antibody (Sigma) Dot blot experiment (FIG. 2).
Liver tissue extract.
ECL Plex anti-mouse CY™5 and CY™3 for Dot blot for ratio-mix experiments (FIG. 8).

### Primary Antibodies:

Rabbit anti-transferrin antibody (Sigma, 1:750 dilution).
Mouse anti-13-tubulin antibody (Sigma, 1:2000 dilution).
Rabbit anti-tubulin, whole antiserum (Sigma, 1:200 dilution).
Chicken anti-TUBB2A antibody (Sigma, 1:3000 dilution).
Rabbit anti-PDGF receptor 0 (28E1), (Cell Signaling, 1:1300 dilution).
Mouse anti-Phospho-PDGF Receptor b (Tyr751) (88H8) (Cell Signaling, 1:1300 dilution).
Rabbit anti-Villin 6884.
Rabbit anti-Villin 6885.

### Traditional Western Blotting:

Tris-Glycine pre-cast gel (12% 15 well or 7.5% polyacryl gel, 26-well), 2% (w/v) ECL™ Advance Blocking Agent in TBS 0.1% TWEEN™, HYBOND™-LFP PVDF membrane HYBOND™-LFP, 0.1% TBS TWEEN™ 20 or TBS wash buffers and Amersham ECL™, ECL™ Plus (anti mouse, rabbit or chicken HRP secondary antibody) or ECL™ Plex (anti-rabbit CY5 secondary antibody) Western blotting detection systems. Probing, washing and detection were performed according to manufactures instructions.

### PLA Western Blotting:

Tris-Glycine pre-cast gel (12% 15 well or 7.5% polyacryl gel, 26-well), 3% (w/v) BSA blocking agent in TBS, 0.1% TWEEN™ 20, 100n/ml salmon sperm DNA. PLA buffer: TBS buffer, 0.5 mg/ml BSA, 5 μg/ml salmon sperm DNA, 5 mM EDTA, 0.05% TWEEN™ 20. Oligo ligation/hybrid buffer: 150/190 mM NaCl, 0.25 mg/ml BSA, 0.05% TWEEN™ 20, 0.5 mM ATP.

T4 ligase buffer (-DTT): 10 mM Tris-Ac, 10 mM MgAc, 50 mM KAc.
RCA buffer: 0.125 mM each of dNTPs, 0.25 mg/ml BSA, 0.05% TWEEN™ 20.
phi29 polymerase buffer: 50 mM Tris-HCl, 10 mM MgCl2, 10 mM (NH4)2SO4, PH 7.5.
Detection buffer: 2×SSC, 0.25 mg/ml BSA, 5 μg/ml salmon sperm DNA, 0.05% TWEEN™ 20.
PLA probe preserving buffer: 1×PBS, 0.05% NaN₃, 0.2 mg/ml BSA.
Anti-mouse, rabbit or chicken secondary PLA probe antibodies and oligo sequences:

PLA probe sequence 1 (SEQ ID NO: 1):
antibody-5′-AAA AAA AAA ATA TGA CAG AAC-
TA GAC ACT CTT-3′ conjugate.
PLA probe sequence 2 (SEQ ID NO: 2):
antibody-5′-AAA AAA AAA AGA CGC TAA TAG
TTA AGA CGC TTU UU-3′ conjugate.
Backbone oligo (SEQ ID NO: 3):
5′-CTA TTA GCG TCC AGT GAA TGC GAG TCC GTC
TAA GAG AGT AGT ACA GCA GCC GTC AAG AGT GTC TA-3′
Splint oligo (SEQ ID NO: 4):
5′-GTT CTG TCA TAT TTA AGC GTC TTA A-3′
Detection probe sequence (SEQ ID NO: 5):
CAG TGA ATG CGA GTC CGT CT
FITC-detection probe (SEQ ID NO: 6):
FITC-AAA AAA CAG TGA ATG CGA GTC CGT
CT.
HRP-detection probe (SEQ ID NO: 7):
HRP-AAA AAA AAA CAG TGA ATG CGA GTC
CGT CT.
(HRP with the diameter of 5 nm, oligo length
about 9.5 nm)
CY ™5-detection probe (SEQ ID NO: 6):
CY ™5-AAA AAA CAG TGA ATG CGA GTC
CGT CT.
CY ™3-detection probe (SEQ ID NO: 6):
CY ™3-AAA AAA CAG TGA ATG CGA GTC
CGT CT.

### PLA Western Probing Protocol:

Incubate primary antibody with gentle orbital rotation in a 5-ml plastic tube chamber at 4° C. overnight. Rinse the membrane once in a plastic container, and then wash 3×9 min with 10 ml TBST in a 15-ml tube by gentle rocking at room temperature. Incubate PLA minus and PLA plus probes in 1×PLA buffer (Olink) containing 0.5% goat serum gentle orbital rotation in a 5-ml plastic tube chamber at room temperature for 1 hour (total volume 1700 μl). Briefly rinse membrane twice with TBS-T. Wash with excess TBS-T, 2×9 min using 15 ml tube with 10 ml washing buffer by gentle rocking at room temperature. Perform backbone/splint oligos hybridization and ligation by incubating 90 nM backbone and splint oligos, 0.05 U/μl T4 ligase in 1× ligation/hybridization buffer (Olink) and 1× ligase buffer (without DTT) (Olink) at 37° C. for 40 minutes during orbital rotation in a 5 ml plastic tube chamber. Briefly rinse once in TBS-T. Wash with 10 ml TBS-T in a 15-ml tube by rocking, 2×6 minutes. Perform rolling circle amplification (RCA) by incubating 0.08 U/μl Phi29 DNA polymerase in 1×Phi29 polymerase buffer and 1×RCA buffer (Olink) at 37° C. for 1 hour during orbital rotation in a 5 ml plastic tube chamber. Briefly rinse once in TBS-T. Wash with 10 ml TBST in a 15 ml tube by gentle rocking once for 6 minutes. Hybridize HRP- or CY™ dye labeled detection probe to RCA product by incubating 5 nM (ECL-readout) or 15 nM (fluorescent-readout) detection probe in 1× detection buffer (Olink) containing 2.5% formamide at 37° C. for 30 minutes during orbital rotation in a 5 ml plastic tube chamber. Briefly rinse twice in TBS-T. Wash with excess TBS-T, 3×9 minutes in a 15 ml tube with 10 ml washing buffer by gentle rocking at room temperature.

### Detection and Image Analysis:

Chemiluminescent (ECL) detection was made by film exposure followed by digitalization of the film using ImageScanner III (GE Healthcare) and fluorescent CY™5 signals were captured by using a fluorescent imager (TYPHOON™ 9410, GE Healthcare). Digital images were then analyzed by using ImageQuant™ TL image analysis software (GE Healthcare) and the signals were quantified.

### Results

Dot blot membranes with a dilution series ranging between 100 ng and 6 pg rabbit anti-transferrin antibody was probed with secondary ECL™ Plex CY™5 antibody (FIG. 2A) or PLA CY™5 probe (FIG. 2B). Signal intensity for CY™5 signals was increased 30 fold for PLA Western compared to ECL™ Plex fluorescent Western blotting using the same intensity setting during scanning with TYPHOON™ Imager (FIG. 2C). Thus, increased fluorescent signal amplification was observed with PLA Western dot blot.

Western blot membranes with a dilution series of human fibroblast cell lysate ranging between 1×10⁵ to 160 cells were probed with mouse anti tubulin primary antibody and anti-mouse secondary HRP antibody (FIG. 3A) or PLA HRP probe (FIG. 3B). The membranes were incubated with ECL reagent and exposed to film for 3 minutes simultaneously (FIG. 3). Increased chemiluminescence signal amplification and improved detection limit was observed with PLA Western.

Human fibroblast cell lysate was applied to Western blotting and the membranes were probed with rabbit anti-tubulin (FIG. 4A, panel 1) or chicken anti-tubulin (FIG. 4A, panel 2) and detected by traditional ECL Western blotting. Both these anti-tubulin primary antibodies showed unspecific protein detection when used separately by traditional ECL Western blotting. When tubulin instead was detected by dual recognition PLA Western (ECL read-out) using a combination of rabbit anti-tubulin and chicken anti-tubulin (FIG. 4A, panel 3) the unspecific detection seen in panel 1 and 2 was removed. In another experiment, tissue samples from liver were applied to Western blotting. The membrane was probed with single PLA Western recognition using either 6884 (FIG. 4B, panel 1) or 6885 (FIG. 4B, panel 2) rabbit anti-Villin primary antibody showing unspecific detection of protein bands. When the membrane was probed with dual PLA Western recognition using both anti-Villin 6884 and 6885 primary antibody (FIG. 4B, panel 3) the unspecific detection was removed. Increased specificity was observed using dual recognition PLA Western compared to single recognition PLA Western (FIG. 4).

Non-stimulated and stimulated human fibroblast cells were applied to Western blotting. One membrane was probed with rabbit anti-PDGFRβ and mouse anti-phosphorylated PDGFRβ showing receptor signal and weak phosphorylation signal separately using traditional ECL Western blotting (FIG. 5A). The other membrane was probed using dual recognition PLA Western using rabbit anti-PDGFRβ and mouse anti-phosphorylated PDGFRβ showing a single enhanced signal for phosphorylated receptor (FIG. 5B). Single detection of protein modification was demonstrated with PLA Western.

To investigate how many ratios of Cy5:Cy3 that could be resolved using a fluorescent Imager, ratios ranging from 10:0 to 0:10 (eleven steps, 10% difference) of ECL Plex anti-mouse Cy5 and Cy3 was mixed and spotted onto a PVDF membrane and scanned at optimal intensity setting (strongest signal just below saturation) in each channel. The results show that at least 6 different ratios of Cy5:Cy3 could be resolved using the Typhoon Imager (FIG. 8).

All patents, patent publications, and other published references mentioned herein are hereby incorporated by reference in their entireties as if each had been individually and specifically incorporated by reference herein. While preferred illustrative embodiments of the present invention are described, one skilled in the art will appreciate that the present invention can be practiced by other than the described embodiments, which are presented for purposes of illustration only and not by way of limitation. The present invention is limited only by the claims that follow.

1. A method for detecting a biomolecular feature in a sample on a porous surface format, said method comprising:
a) providing a sample containing said bio molecular feature onto said porous surface;
b) providing a proximity probe pair, each probe comprising a binding moiety with affinity for a different binding site on said bio molecular feature and an oligonucleotide acting as a reactive functionality (reactive oligonucleotide), coupled thereto;
c) binding said proximity probes to their respective binding sites on said biomolecular feature through the binding moiety;
d) adding a splint oligonucleotide and a backbone oligonucleotide which are complementary to the reactive oligonucleotide pair, and allowing them to hybridize, thereby bringing the ends of the backbone and splint oligonucleotides in direct contact;
e) ligating, using a DNA ligase, the hybridized DNA oligonucleotides to create a circularized DNA molecule wherein said circularized DNA molecule is formed from the backbone and splint oligonucleotides only when said probes in said proximity probe pair bind sufficiently close to each other on said bio molecular feature;
f) elongating one of the reactive oligonucleotides by isothermal amplification using the circularized DNA molecule as template, thereby creating a localized amplification product; and
g) detecting the presence and quantity of said biomolecular feature using a detection oligonucleotide complementary to the amplification product;
wherein said biomolecular feature is a protein, a protein complex, or a modified protein such as a phosphorylated protein.

2. The method of claim 1, wherein the binding moieties are antibodies and said antibodies each bind to said biomolecular feature via the aid of one or two further antibody/antibodies having direct binding specificity for the biomolecular feature, and wherein the binding moieties are directed against the Fc portion or conjugated haptens of the further antibody/antibodies.

3. The method of claim 1, wherein said isothermal amplification is rolling circle amplification.

4. The method of claim 1, wherein said isothermal amplification is performed using Phi29 DNA polymerase.

5. The method of claim 1, wherein the binding moieties of the proximity probes have direct specificity for the bio molecular feature and are selected from a protein, such as a monoclonal or polyclonal antibody, lectin, soluble cell surface receptor, combinatorially derived protein from phage display or ribosome display, peptide, carbohydrate, nucleic acid, such as an aptamer, or combinations thereof.

6. The method of claim 1, wherein said sample is a homogenized tissue or cell lysate, a body fluid, or cell culture supernatant.

7. The method of claim 1, wherein the binding between the proximity probes and the bio molecular feature as well as the detection are performed directly in an electrophoretic gel.

8. The method of claim 1, wherein the sample containing said bio molecular feature is first separated by gel electrophoresis and then transferred from the electrophoretic gel onto a suitable blotting membrane, such as a nitrocellulose or PVDF membrane, before binding and detection with the proximity probes.

9. The method of claim 1, wherein the sample containing said bio molecular feature is directly spotted onto a suitable porous membrane, such as a nitrocellulose or PVDF membrane, before binding and detection with the proximity probes.

10. The method of claim 1, wherein said detection oligonucleotides are fluorescently labeled and the detection is performed by fluorescence read-out.

11. The method of claim 1, wherein said detection oligonucleotides are labeled with an HRP enzyme and the detection is performed by ECL read-out.

12. A method for multiplexed detection of two or more target biomolecular feature in a sample on a porous membrane format, said method comprising:
a) providing a sample containing said bio molecular features onto a porous surface;
b) for each of the target bio molecular feature, providing a proximity probe pair, each probe comprising a binding moiety with affinity for a different binding site on said bio molecular feature and an oligonucleotide acting as a reactive functionality (reactive oligonucleotide), coupled thereto; wherein when both proximity probes bind to the same bio molecular feature in proximity, a circularized DNA molecule is formed and an amplification product (i.e., rolling circle product) can be generated in subsequent steps, further wherein the amplification product from each proximity probe pair carries at least one unique detection sequence site, which is unique from a detection sequence site in an amplification product for a different target bio molecular feature;
c) binding said proximity probe pairs to their respective binding sites on each of said bio molecular features through the binding moiety,
d) forming a circularized DNA molecule when said probes in said proximity probe pair bind sufficiently close to each other on a bio molecular feature,
e) elongating one of the reactive oligonucleotides by isothermal amplification using the circularized DNA molecule as template, thereby creating a localized amplification product;
f) providing, for each target bio molecular feature, a detection oligonucleotide mixture based on a sequence which is complimentary to said unique detection sequence site on the amplification product; wherein the detection oligonucleotide mixture for said target bio molecular feature contains a defined ratio of species with identical sequence but labeled with different fluorescent dyes such that during detection, a unique signal ratio is associated with the amplification product for said bio molecular feature;
g) pooling the detection oligonucleotide mixtures for all the target bio molecular features and hybridizing said detection oligonucleotide mixtures with the amplification products;
h) detecting the signal from each label for each of the two or more different labels;
i) generating a signal gain calibration for the different labels using a predefined standard sample present on a defined and spatially separated region of the reaction volume; and
j) calculating the ratio of labels for each location using said signal gain calibration and detecting the target bio molecular feature of interest based on the detection of the unique signal ratios;
wherein said bio molecular features are proteins, protein complexes, or modified proteins such as phosphorylated proteins.

13. The method for multiplexed detection of two or more target biomolecular features of claim 12, further comprising: for each of the target bio molecular features, providing one splint oligonucleotide and one backbone oligonucleotide which are complementary to the reactive oligonucleotides, thus a circularized DNA molecule is formed through hybridization and ligation of these oligonucleotides, provided both proximity probes bind to the same bio molecular feature in proximity.

14. The method of claim 12, wherein the binding moieties are antibodies and said antibodies each bind to said bio molecular feature via the aid of one or two further antibody/antibodies having direct binding specificity for the bio molecular feature, and wherein the binding moieties are directed against the Fc portion or conjugated haptens of the further antibody/antibodies.

15. The method of claim 12, wherein said isothermal amplification is rolling circle amplification.

16. The method of claim 15, wherein said isothermal amplification is performed using Phi29 DNA polymerase.

17. The method of claim 12, wherein the binding moieties of the proximity probes have direct specificity for the biomolecular feature and are selected from a protein, such as a monoclonal or polyclonal antibody, lectin, soluble cell surface receptor, combinatorially derived protein from phage display or ribosome display, peptide, carbohydrate, nucleic acid, such as an aptamer, or combinations thereof.

18. The method of claim 12, wherein said sample is a homogenized tissue or cell lysate, a body fluid, or cell culture supernatant.

19. The method of claim 12, wherein the binding between the proximity probe pairs and the bio molecular features as well as the detection are performed directly in an electrophoretic gel.

20. The method of claim 12, wherein the sample containing said bio molecular features is first separated using gel electrophoresis and then transferred from the electrophoretic gel onto a suitable blotting membrane, such as a nitrocellulose or PVDF membrane, before binding and detection with the proximity probes.

21. The method of claim 12, wherein the sample containing the bio molecular feature is spotted directly onto a suitable membrane, such as a nitrocellulose or PVDF membrane, before binding and detection with the proximity probes.

22. The method of claim 12, wherein said detecting step is performed by taking multiple scans/images at different excitation wavelength/emission filter combinations.

23. The method of claim 12, wherein said detecting step is performed by multi-spectral imaging (i.e. by recoding complete emission spectra in each pixel).

24. A kit for proximity ligation assay based Western blot analysis, comprising:
a proximity probe pair, each probe comprising a binding moiety with affinity for a different binding site on a bio molecular feature and an oligonucleotide acting as a reactive functionality (reactive oligonucleotide), coupled thereto;
a splint oligonucleotide and a backbone oligonucleotide which are complementary to the reactive oligonucleotide pair;
a detection oligonucleotide; and
one or more optimized buffers, and protocols.

25. The kit of claim 24, wherein said detection oligonucleotide is labeled.

26. The kit of claim 24, wherein the binding moieties are antibodies and said antibodies each bind to said bio molecular feature via the aid of one or two further antibody/antibodies having direct binding specificity for the bio molecular feature, and wherein the binding moieties are directed against the Fc portion or conjugated haptens of the further antibody/antibodies.

27. The kit of claim 24, further comprises a DNA ligase and an enzyme for isothermal amplification, such as rolling circle amplification.

28. The kit of claim 24, for use in analysis of two or more target bio molecular feature, wherein the kit comprises:
for each of the target bio molecular features,
a proximity probe pair, each probe comprising a binding moiety with affinity for a different binding site on the bio molecular feature and an oligonucleotide acting as a reactive functionality (reactive oligonucleotide), coupled thereto;
a splint oligonucleotide and a backbone oligonucleotide which are complementary to the reactive oligonucleotide pair, thus a circularized DNA molecule can be formed and an amplification product can be generated during the reaction process, provided both proximity probes bind to the same bio molecular feature in proximity;
a detection oligonucleotide mixture based on a sequence which is complimentary to a unique detection sequence site on the amplification product; wherein the detection oligonucleotide mixture for each target substrate contains a defined ratio of species with identical sequence but labeled with different fluorescent dyes such that during detection, a unique signal ratio is associated with the amplification product for each bio molecular feature; and
the kit further includes a standard sample for calibration of fluorescent dye signal gains; one or more optimized buffers; and protocols.

29. The kit of claim 28, further comprising a DNA ligase and an enzyme for isothermal amplification, such as rolling circle amplification.

30. The kit of claim 28, wherein the binding moieties are antibodies and said antibodies each bind to said bio molecular feature via the aid of one or two further antibody/antibodies having direct binding specificity for the bio molecular feature, and wherein the binding moieties are directed against the Fc portion or conjugated haptens of the further antibody/antibodies.

31. The kit of claim 28, wherein the binding moieties have direct specificity for the bio molecular feature and are selected from a protein, such as a monoclonal or polyclonal antibody, lectin, soluble cell surface receptor, combinatorially derived protein from phage display or ribosome display, peptide, carbohydrate, nucleic acid, such as an aptamer, or combinations thereof.
